SEQUENCE LISTING (2 sequences)

SEQ ID NO: 1 - protein, 84 aa, Homo sapiens
SVSEIQLMHNLGKHLNSMERVEWLRKKLQDVHNFVALGAPLAPRDAGSQRPRKKEDNVLVESHEKSLGEADKADVNVLTKAKSQ

SEQ ID NO: 2 - protein, 34 aa, Homo sapiens
SVSEIQLMHNLGKHLNSMERVEWLRKKLQDVHNF

(19) United States
(12) Patent Application Publication
(10) Pub. No.: US 2023/0190880 A1
(43) Pub. Date: Jun. 22, 2023

(54) Formulations of Human Parathyroid Hormone (PTH) and Methods for Producing Same

(71) Applicant: Sichuan Luzhou Buchang BIO-Pharmaceutical Co., Ltd., Luzhou (CN)

(72) Inventors: Huan Wang, Beijing (CN); Lei Shi, Beijing (CN); Xiaowen Li, Beijing (CN); Linhong Zhang, Beijing (CN); Xiang Guo, Beijing (CN); Chun Yang, Beijing (CN); Tiansheng Li (deceased), Newbury Park, CA (US)

(21) Appl. No.: 17/916,001

(22) PCT Filed: Mar. 30, 2020
(86) PCT No.: PCT/CN2020/082163

§ 371 Date: Sep. 29, 2022

(51) Int. Cl. A61K 38/29; A61K 9/08; A61K 9/00; A61K 47/26

(52) U.S. Cl. CPC A61K 38/29; A61K 9/08; A61K 9/0019; A61K 47/26

(57) ABSTRACT

Stable liquid pharmaceutical formulations comprising human parathyroid hormone are provided herein. In various embodiments, the stable liquid pharmaceutical formulations comprise human parathyroid hormone (huPTH); a buffering agent to maintain the pH range of the solution from 3 to 6; one or more stabilizing agents selected from the group consisting of sugars, salts, surfactants, proteins, chaotropic agents, lipids, and amino acids; a tonicity modifier; water; and optionally a parenterally acceptable preservative; and wherein said solution is sterile and ready for parenteral administration to a human patient.

### CROSS-REFERENCE TO RELATED APPLICATION

The present application is a U.S. National Phase Entry of International PCT Application No. PCT/CN2020/082163 having an international filing date of Mar. 30, 2020, which is incorporated herein by reference in its entirety.

### SEQUENCE LISTING

The instant application contains a Sequence Listing in the form of a “paper copy” (PDF File) and a file containing the referenced sequences (SEQ ID NO: 1-SEQ ID NO: 2) in computer readable form (ST25 format text file) which is submitted herein. The Sequence Listing is shown using standard three letter code for amino acids, as defined in 37 C.F.R. 1.822.

### BACKGROUND OF THE INVENTION

Parathyroid hormone (PTH), also called parathormone or parathyrin, is a hormone secreted by the parathyroid glands that regulates the serum calcium through its effects on bone, kidney, and intestine. PTH influences bone remodeling, which is an ongoing process in which bone tissue is alternately resorbed and rebuilt over time. PTH is secreted in response to low blood serum calcium levels. PTH indirectly stimulates osteoclast activity within the bone matrix in order to release more ionic calcium into the blood to elevate a low serum calcium level. Disorders that yield too little or too much PTH, such as hypoparathyroidism, hyperparathyroidism, and paraneoplastic syndromes can cause bone disease, hypocalcemia, and hypercalcemia.

PTH is a polypeptide containing 84 amino acids, which is a prohormone. It has a molecular mass around 9500 Da. Studies in humans with certain forms of PTH have demonstrated an anabolic effect on bone and have prompted significant interest in its use for the treatment of osteoporosis and related bone disorders. Using the N-terminal 34 amino acids of the bovine and human hormone, it has been demonstrated in humans that parathyroid hormone enhances bone growth particularly when administered in pulsatile fashion by the subcutaneous route. Teriparatide (PTH 1-34) (FORTED®) is approved in the United States for treatment of osteoporosis in those at high risk of fracture including postmenopausal women, men with primary or hypogonadal osteoporosis and men and women with glucocorticoid-associated osteoporosis. A slightly different form of PTH, human PTH (1-38) has shown similar results (Cusano N E et al., J Clin Endocrinol Metab., 98:137-144, 2013).

Unfortunately, like many small molecule therapeutics, PTH is susceptible to degradation by proteases and labile due to degradation. In fact, it is more labile than the traditional small molecules. PTH is highly sensitive to deamidation, clips, aggregation and oxidation primarily at N-terminal amino acids, e.g., methionine residues in the positions 8 and 18 giving rise to oxidized PTH species. Furthermore, it can get deamidated at asparagine residue in position 16. There is a probability of truncation of polypeptide chain at N-terminal and C-terminals due to breakage of peptide bond. All these reactions can significantly hamper the bioactivity of this protein.

Improved formulations of PTH which serve to prevent these adverse reactions are much needed.

### Incorporation by Reference

All publications and patent applications mentioned in this specification are herein incorporated by reference to the same extent as if each individual publication or patent application was specifically and individually indicated to be incorporated by reference.

### SUMMARY OF THE INVENTION

It is an object of the present invention to provide a pharmaceutically useful PTH formulation in the form of a stabilized liquid solution containing a parathyroid hormone (PTH) in a therapeutically effective amount. The solution is storage stable and, in sterile form, may be stored in vials or cartridges ready for parenteral administration in human patients.

In various embodiments, the stable liquid formulations comprise human parathyroid hormone (huPTH) in a concentration of about 100-2000 μg/ml; an acetate or citrate buffer to maintain the pH range of the solution from 3 to 7; a stabilizing agent selected from the group consisting of sugars, salts, surfactants, proteins, chaotropic agents, lipids, and amino acids; water; and optionally a parenteraly acceptable preservative; wherein said solution is sterile and ready for parenteral administration to a human patient.

In one embodiment, the stable liquid formulation comprises huPTH (1 mg/mL), 10 mM sodium citrate, 50 mM L-Met, 10 mM EDTA, 100 mM NaCl, 0.01% polysorbate 20, pH 5.0.

### BRIEF DESCRIPTION OF THE DRAWINGS

FIG. 1 depicts an SEC-HPLC chromatogram comparing a standard reference huPTH sample (top) with a huPTH sample that had undergone forced oxidation (bottom).

FIG. 2 depicts an RP-HPLC chromatogram comparing a standard reference huPTH sample (top) with a huPTH sample that had undergone forced oxidation (bottom).

FIG. 3 depicts an CEX-HPLC chromatogram comparing a standard reference huPTH sample (top) with a huPTH sample that had undergone forced oxidation (bottom).

FIG. 4 depicts an SEC-HPLC chromatogram comparing various huPTH formulations at 25° C. (top) and 37° C. (bottom).

FIG. 5 depicts an RP-HPLC chromatogram comparing various huPTH formulations at 25° C. (top) and 37° C. (bottom).

FIG. 6 depicts an CEX-HPLC chromatogram comparing various huPTH formulations at 25° C. (top) and 37° C. (bottom).

FIG. 7 depicts an SEC-HPLC chromatogram comparing various huPTH formulations at 4° C.

FIG. 8 depicts an RP-HPLC chromatogram comparing various huPTH formulations at 4° C.

FIG. 9 depicts an RP-HPLC chromatogram comparing various huPTH formulations at 25° C. (top) and 37° C. (bottom).

FIG. 10 depicts an RP-HPLC chromatogram comparing various huPTH formulations at 4° C.

FIG. 11 depicts an SEC-HPLC chromatogram comparing various huPTH formulations at 25° C. (top) and 37° C. (bottom).

FIG. 12 depicts an SEC-HPLC chromatogram comparing various huPTH formulations at 4° C.

FIG. 13 depicts an CEX-HPLC chromatogram comparing various huPTH formulations at 25° C. (top) and 37° C. (bottom).

FIG. 14 depicts an CEX-HPLC chromatogram comparing various huPTH formulations at 4° C.

FIG. 15 depicts an RP-HPLC chromatogram comparing various huPTH formulations at 25° C. (top) and 37° C. (bottom).

FIG. 16 depicts an RP-HPLC chromatogram comparing various huPTH formulations at 4° C.

FIG. 17 depicts an SEC-HPLC chromatogram comparing various huPTH formulations at 25° C. (top) and 37° C. (bottom).

FIG. 18 depicts an SEC-HPLC chromatogram comparing various huPTH formulations at 4° C.

FIG. 19 depicts an CEX-HPLC chromatogram comparing various huPTH formulations at 25° C. (top) and 37° C. (bottom).

FIG. 20 depicts an CEX-HPLC chromatogram comparing various huPTH formulations at 4° C.

FIG. 21 depicts an RP-HPLC chromatogram evaluating the effects of Tween 20 vs Tween 80 on huPTH formulations at 37° C.

FIG. 22 is a bar graph depicting the effects of Tween 20 vs Tween 80 on huPTH formulations at 37° C. after 5 days.

FIGS. 23-25 are bar graphs depicting the stability of various formulations at 4° C., 25° C. and 37° C. after one week.

FIGS. 26-28 are bar graphs depicting RP-HPLC evaluation of various huPTH formulations at 4° C., 25° C. and 37° C.

FIGS. 29-31 are bar graphs depicting CEX-HPLC evaluation of various huPTH formulations at 4° C., 25° C. and 37° C.

FIGS. 32-34 are bar graphs depicting SEC-HPLC evaluation of various huPTH formulations at 4° C., 25° C. and 37° C.

FIGS. 35-37 are bar graphs depicting RP-HPLC evaluation of various huPTH formulations at 4° C., 25° C. and 37° C.

FIGS. 38-40 are bar graphs depicting CEX-HPLC evaluation of various huPTH formulations at 4° C., 25° C. and 37° C.

FIGS. 41-43 are bar graphs depicting SEC-HPLC evaluation of various huPTH formulations at 4° C., 25° C. and 37° C.

### DETAILED DESCRIPTION

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. The term “about” also includes the exact value “X” in addition to minor increments of “X” such as “X+0.1” or “X−0.1.” It also is to be understood, although not always explicitly stated, that the reagents described herein are merely exemplary and that equivalents of such are known in the art.

As used herein, certain terms have the following defined meanings.

As used herein, the term “comprising” is intended to mean that the formulations and methods include the recited elements, but do not exclude others. “Consisting essentially of” when used to define formulations and methods, shall mean excluding other elements of any essential significance to the combination when used for the intended purpose. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the formulations of this invention. Embodiments defined by each of these transition terms are within the scope of this invention.

As used herein, the term “aqueous pharmaceutical formulation” or “liquid pharmaceutical formulation” refers to a formulation of a therapeutically effective amount of an active ingredient in water suitable for administration to a patient.

In various embodiments the active ingredient of the pharmaceutical formulation is a biologically active hPTH is selected from the group comprising hPTH (1-34), hPTH (1-37), hPTH (1-38), hPTH (1-41) and hPTH (1-84). In various embodiments, the liquid formulation may incorporate the full length, 84 amino acid form of parathyroid hormone, particularly the human form, hPTH (1-84) (SEQ ID NO: 1),

(SEQ ID NO: 1)
SVSEIQLMHNLGKHLNSMERVEWLRKKLQDVHNFVALGAPLAPRDAGSQR
PRKKEDNVLVESHEKSLGEADKADVNVLTKAKSQ

obtained either recombinantly, by peptide synthesis, or by extraction from human fluid (see, e.g. U.S. Pat. No. 5,208,041, incorporated herein by reference).

In various embodiments, the PTH fragments incorporate at least the first 34 N-terminal residues, such as PTH (1-37), PTH (1-38), PTH (1-41) and PTH (1-34) (SEQ ID NO: 2),

| (SEQ ID NO: 2) | |
|---|---|
| | SVSEIQLMHNLGKHLNSMERVEWLRKKLQDVHNF |

Alternatives in the form of PTH variants incorporate from 1 to 5 amino acid substitutions that improve PTH stability and half-life, such as the replacement of methionine residues at positions 8 and/or 18 with leucine or other hydrophobic amino acid that improves PTH stability against oxidation and the replacement of amino acids in the 25-27 region with trypsin-insensitive amino acids such as histidine or other amino acid that improves PTH stability against protease. These forms of PTH are embraced by the term “parathyroid hormone” as used generically herein. The hormones may be obtained by known recombinant or synthetic methods, such as described in U.S. Pat. No. 4,086,196, incorporated herein by reference.

In various embodiments, the pharmaceutical formulation typically contains about 0.01 mg/mL to about 5 mg/mL of PTH, about 0.1 mg/mL to about 2.5 mg/mL of PTH, or about 0.5 mg/mL to about 1 mg/mL of PTH. In various embodiments, the pharmaceutical formulation contains about 0.25 mg/mL of PTH, about 0.5 mg/mL of PTH, about 1 mg/mL of PTH or about 2 mg/mL of PTH. In various embodiments, the pharmaceutical formulation contains 0.1 mg/mL to 1 mg/mL of PTH. In one embodiment, the formulation contains 1 mg/mL of PTH.

The pharmaceutical formulations are generally formulated appropriately for immediate use. In various embodiments when the pharmaceutical formulation is not to be administered immediately, the PTH can be formulated in a formulation suitable for storage. One such formulation is a lyophilized formulation of the PTH together with a suitable stabilizer. Alternatively, the PTH can be formulated for storage in a solution with one or more suitable stabilizers. Any such stabilizer known to one of skill in the art without limitation can be used. In various embodiments, stabilizers suitable for lyophilized preparations include, but are not limited to, sugars, salts, surfactants, proteins, chaotropic agents, lipids, and amino acids. In various embodiments, stabilizers suitable for liquid preparations include, but are not limited to, sugars, salts, surfactants, proteins, chaotropic agents, lipids, and amino acids.

As used herein, the term “buffer” or “buffer solution” refers to a generally aqueous solution comprising a mixture of an acid (usually a weak acid, e.g. acetic acid, citric acid, imidazolium form of histidine) and its conjugate base (e.g. an acetate or citrate salt, for example, sodium acetate, sodium citrate, or histidine) or alternatively a mixture of a base (usually a weak base, e.g. histidine) and its conjugate acid (e.g. protonated histidine salt). The pH of a “buffer solution” will change very only slightly upon addition of a small quantity of strong acid or base due to the “buffering effect” imparted by the “buffering agent”. The phrase “buffer system” means a mixture containing at least two buffers. In various embodiments of the invention, the buffering agent incorporated into the pharmaceutical formulation includes any acid or salt combination which is pharmaceutically acceptable and capable of maintaining the aqueous solution at a pH range of 3 to 7. In various embodiments, the buffer maintains a pH of about 3.0 to about 7.0. In various embodiments, the buffer maintains a pH of about 3.0, a pH of about 4.0, a pH of about 5.0, a pH of about 6.0, or a pH of about 7.0.

Any buffer that is capable of maintaining the pH of the formulation at any pH or within any pH range provided above is suitable for use in the pharmaceutical formulations of the present disclosure, provided that it does not react with other components of the formulation, cause visible precipitates to form, or otherwise cause the active ingredient to become chemically destabilized. Examples of suitable buffers are well known in the literature (see, for example, Allen Jr, Loyd V, ed. (2012) Remington: The Science and Practice of Pharmacy, 22^{nd} ed., Pharmaceutical Press). In various embodiments, the buffer used in the pharmaceutical formulation comprises a component selected from the group consisting of succinate, citrate, malate, edentate, histidine, acetate, adipate, aconitate, ascorbate, benzoate, carbonate, bicarbonate, maleate, glutamate, lactate, phosphate, and tartarate, or a mixture of these buffers.

The concentration of the buffer is selected so that pH stabilization as well as sufficient buffering capacity is provided. In various embodiments, buffer systems are acetate or citrate sources. In various embodiments, the buffer system is a citrate source. In various embodiments, the buffer is present at about 5 mM, at about 10 mM, at about 15 mM or about 20 mM. In other embodiments, the buffer is present in the formulation at a concentration of from 0.5 to 100 mM, from 0.75 to 50 mM, from 1 to 20 mM, or from 10 to 20 mM. In one embodiment, the buffer is present at about 10 mM. In one embodiment, the buffer is citrate present at 10 mM.

In various embodiments of the invention, the stabilizing agent incorporated in the pharmaceutical formulation is selected from the group consisting of: sugars, salts, surfactants, proteins, chaotropic agents, lipids, and amino acids. In various embodiments, the stabilizing agent is selected from the group consisting of a polyol which includes a saccharide, preferably a monosaccharide or disaccharide, e.g., mannitol, glycine, glycerol, sorbitol or inositol and a polyhydric alcohol such as glycerine or propylene glycol or mixtures thereof; a chelator selected from the group of EDTA, DTPA or EGTA; an amino acid(s) selected from the group of proline, alanine, arginine, asparagines, aspartic acid, cysteine, glutamine, glutamic acid, glycine, histidine, isoleucine, leucine, lysine, methionine, phenylalanine, serine, threonine, tryptophan, tyrosine, and valine. Examples of suitable stabilizing agents have been described extensively in the art (see, for example, Allen Jr, Loyd V, ed. (2012) Remington: The Science and Practice of Pharmacy, 22^{nd} ed., Pharmaceutical Press).

In various embodiments of the invention, the stabilizing agent incorporated into the pharmaceutical formulation is a surfactant. A “surfactant” as used herein refers to an amphiphilic compound, i.e. a compound containing both hydrophobic groups and hydrophilic groups which lowers the surface tension (or interfacial tension) between two liquids or between a liquid and a solid. A “non-ionic surfactant” has no charged groups in its head. Examples of “non-ionic surfactants” include e.g. polyoxyethylene glycol alkyl ethers, such as octaethylene glycol monododecyl ether, pentaethylene glycol monododecyl ether; polyoxypropylene glycol alkyl ethers; glucoside alkyl ethers, such as decyl glucoside, lauryl glucoside, octyl glucoside; polyoxyethylene glycol octylphenol ethers, such as triton X-100; polyoxyethylene glycol alkylphenol ethers, such as nonoxynol-9; glycerol alkyl esters, such as glyceryl laurate; polyoxyethylene glycol sorbitan alkyl esters, such as polysorbate; sorbitan alkyl esters, such as spans; cocamide MEA, cocamide DEA, dodecyldimethylamine oxide; block copolymers of polyethylene glycol and polypropylene glycol, such as poloxamers; and polyethoxylated tallow amine (POEA). In various embodiments, the pharmaceutical formulations of the present invention can contain one or more of these surfactants in combination. In various embodiments, the non-ionic surfactants for use in the pharmaceutical formulations are selected from the group consisting of polysorbates such as polysorbate 20, 40, 60 or 80, and the concentration of the non-ionic surfactant is in the range of 0.01 to 0.08% (w/v), 0.015 to 0.06% (w/v), or 0.02 to 0.04% (w/v), relative to the total volume of the formulation. In one embodiment, the non-ionic surfactant is polysorbate 20 (i.e. Tween 20) with a concentration of 0.01% (w/v), relative to the total volume of the formulation.

In various embodiments of the invention, the chelator incorporated into the stable liquid formulation is selected from the group consisting of EDTA, DTPA or EGTA. In various embodiments, the chelator is EDTA at a concentration of 10 mM.

In various embodiments of the invention, the amino acid incorporated into the stable liquid formulation is selected from the group consisting of proline, alanine, arginine, asparagines, aspartic acid, cysteine, glutamine, glutamic acid, glycine, histidine, isoleucine, leucine, lysine, methionine, phenylalanine, serine, threonine, tryptophan, tyrosine, and valine. In various embodiments, the amino acid is L-methionine at a concentration of 50 mM.

In various embodiments of the invention, the formulation of the present disclosure contains a physiologically acceptable tonicity modifier. The phrase “tonicity modifier” means a pharmaceutically acceptable inert substance that can be added to the formulation to adjust the tonicity of the formulation. Tonicity modifiers suitable for this invention include, but are not limited to, sodium chloride, potassium chloride, mannitol, sucrose, dextrose, sorbitol, glycerin and other pharmaceutically acceptable tonicity modifier. When a tonicity agent is present, it is preferably present in an amount sufficient to make the liquid formulation approximately isotonic with bodily fluids (i.e., about 270 to about 300 mOsm/L) and suitable for parenteral injection into a mammal, such as a human subject, into dermal, subcutaneous, or intramuscular tissues or IV. Isotonicity can be measured by, for example, using a vapor pressure or ice-freezing type osmometer.

In various embodiments of the invention, the pharmaceutical formulation will contain a tonicity modifier selected from the group consisting of potassium chloride, calcium chloride, sodium chloride, sodium phosphate, potassium phosphate and sodium bicarbonate, and the concentration will be in the range of 10 to 200 mM, 20 to 150 mM, or 30 to 100 mM. In one embodiment, the tonicity modifier is sodium chloride at a concentration of 100 mM.

In various embodiments of the invention, the stable liquid formulation may also include a parenterally acceptable preservative selected from the group consisting of cresols, benzyl alcohol, phenol, benzalkonium chloride, benzethonium chloride, chlorobutanol, phenylethyl alcohol, methyl paraben, propyl paraben, thimerosal and phenylmercuric nitrate and acetate.

As used herein, the terms “sterile water” or “water for injection” refer to a sterile, nonpyrogenic preparation of water for injection which contains no bacteriostat, antimicrobial agent or added buffer. In general, the osmolar concentration of additives totals at least 112 mOsmol/liter (two-fifths of the normal osmolarity of the extracellular fluid −280 mOsmol/liter).

The pharmaceutical formulations of the present invention are suitable for parenteral administration. As used herein, “parenteral administration” of a pharmaceutical formulation includes any route of administration characterized by physical breaching of a tissue of a subject and administration of the pharmaceutical formulation through the breach in the tissue, thus generally resulting in the direct administration into the blood stream, into muscle, or into an internal organ. Parenteral administration thus includes, but is not limited to, administration of a pharmaceutical formulation by injection of the formulation, by application of the formulation through a surgical incision, by application of the formulation through a tissue-penetrating non-surgical wound, and the like. In particular, parenteral administration is contemplated to include, but is not limited to, subcutaneous injection, intraperitoneal injection, intramuscular injection, intrasternal injection, intravenous injection, intraarterial injection, intrathecal injection, intraventricular injection, intraurethral injection, intracranial injection, intrasynovial injection or infusions; or kidney dialytic infusion techniques. The present invention is related to a stable aqueous pharmaceutical formulation for use in a pre-filled syringe, vial, cartridge, or pen.

The pharmaceutical formulations are generally formulated as sterile, substantially isotonic and in full compliance with all Good Manufacturing Practice (GMP) regulations of the U.S. Food and Drug Administration. Preferably, a therapeutically effective dose of the polypeptide formulations described herein will provide therapeutic benefit without causing substantial toxicity.

In one embodiment, the stable liquid formulation comprises huPTH (1 mg/mL), 10 mM sodium citrate, 50 mM L-Met, 10 mM EDTA, 100 mM NaCl, 0.01% polysorbate 20, pH 5.0

The examples which follow are illustrative of the invention and are not intended to be limiting.

### EXAMPLE 1

### Formulation Stability Method Development

In the development of a method for evaluating stability, a test formulation of huPTH (1 mg/mL PTH) underwent forced oxidation with 0.01% H₂O₂ and was then subjected to SEC-HPLC, RP-HPLC, and CEX-HPLC as described in the Additional Methods section below.

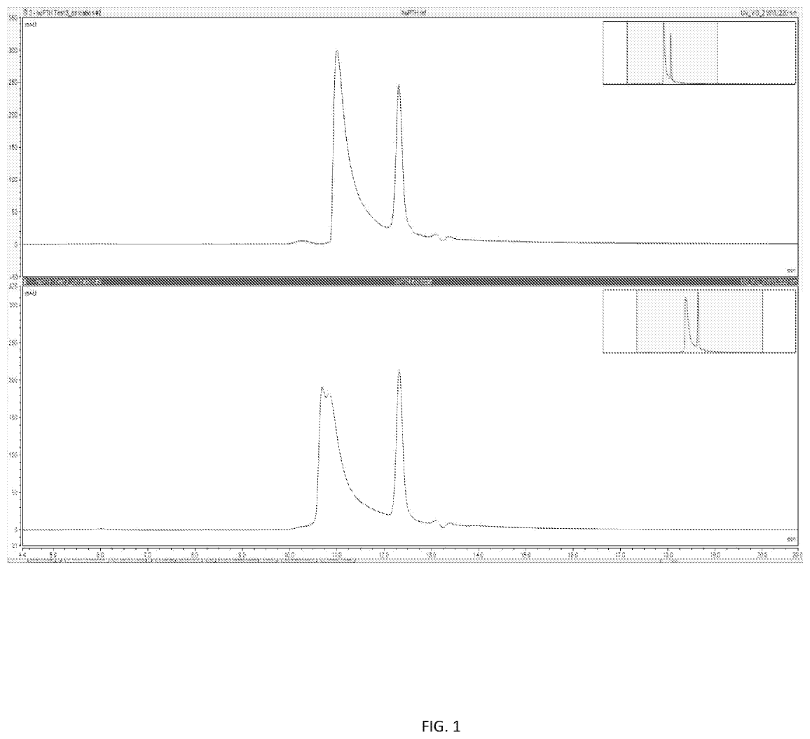

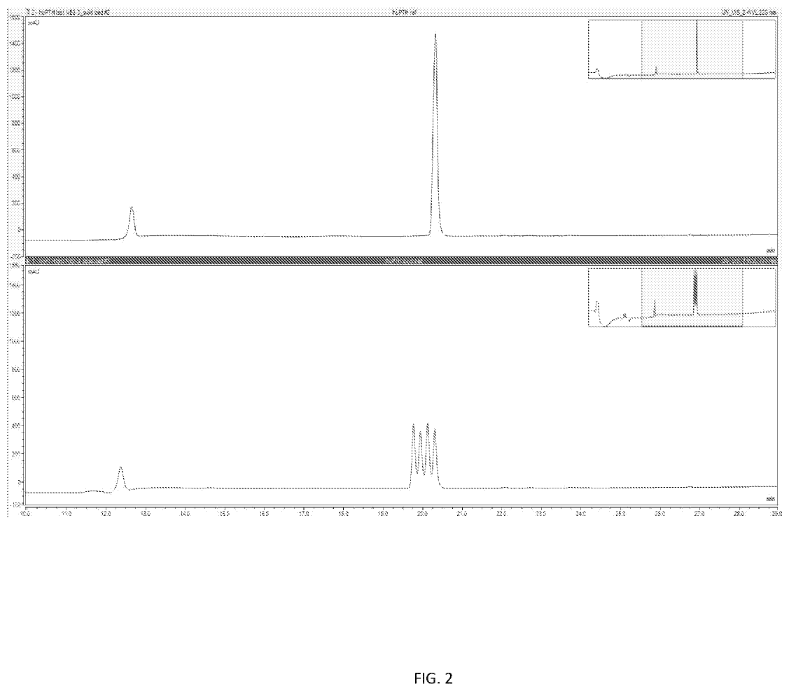

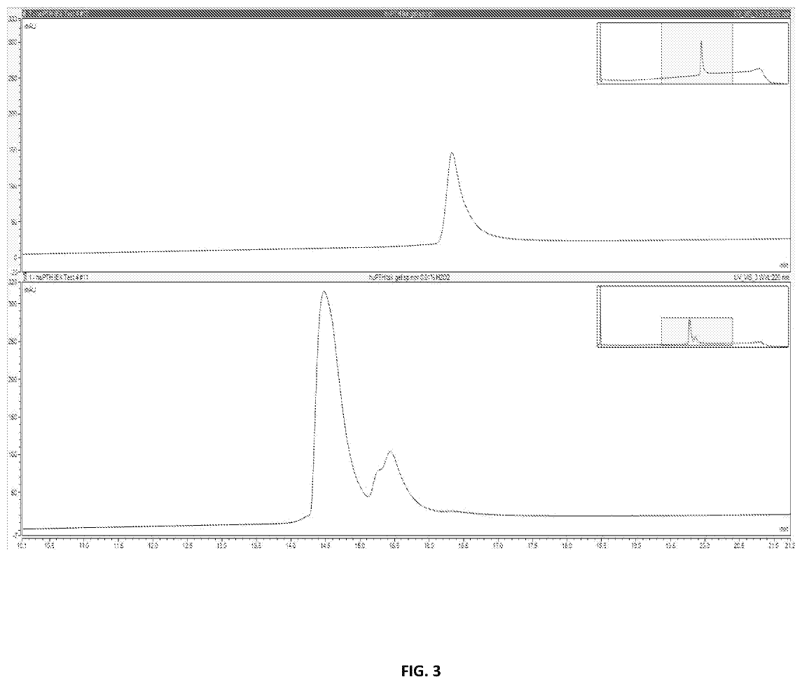

The results of the SEC-HPLC analysis is depicted in FIG. 1. As depicted in FIG.1, the SEC main peak shifts to an earlier elution time, indicating aggregation caused by forced oxidation. The results of the RP-HPLC analysis is depicted in FIG. 2. As depicted in FIG. 2, the RP main peak splits into four eluting peaks, indicating more hydrophilic species of PTH caused by forced oxidation. The results of the CEX-HPLC analysis is depicted in FIG. 3. As depicted in FIG. 3, the main peak shifts to an earlier elution time, indicating less positively charged species such as deamindated species caused by forced oxidation.

### EXAMPLE 2

### Screening Studies of pH and Buffers

This example demonstrates the effects of pH and varying buffer conditions on the stability of liquid huPTH formulations. Recombinant huPTH (1 mg/mL) having the amino acid sequence set forth in SEQ ID NO: 2 was used as an active ingredient in the formulations. Table 1 provides a summary of the formulations that were tested in this example.

TABLE 1

10 mM sodium acetate, 140 mM NaCl, pH 4.0
10 mM sodium acetate, 140 mM NaCl, pH 5.0
10 mM citrate, 140 mM NaCl, pH 5.0
10 mM sodium phosphate, 140 mM NaCl, pH 6.0
10 mM histidine, 140 mM NaCl, pH 6.0
10 mM sodium phosphate, 140 mM NaCl, pH 7.0
10 mM Tris, 140 mM NaCl, pH 7.0
10 mM Tris, 140 mM NaCl, pH 8.0
10 mM glycine, 140 mM NaCl, pH 9.0

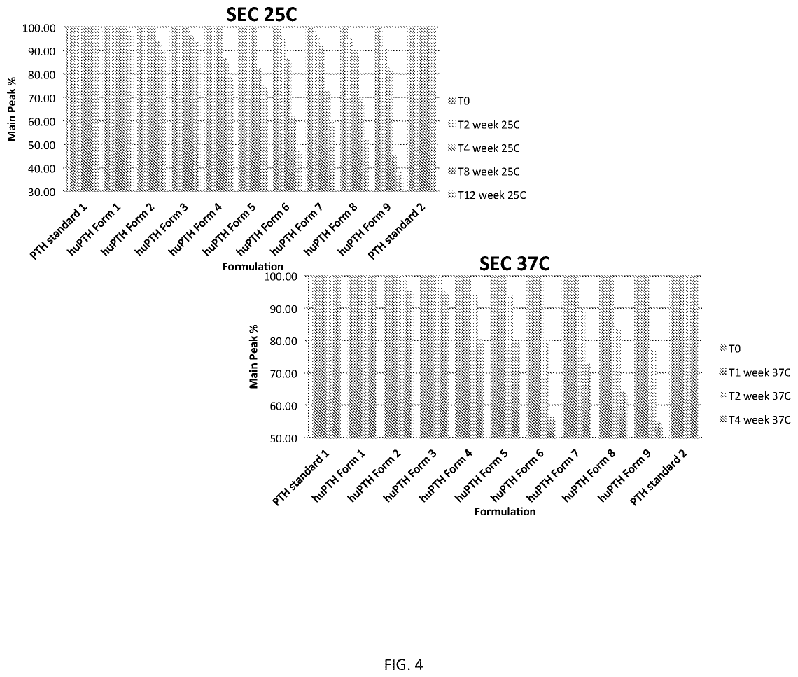

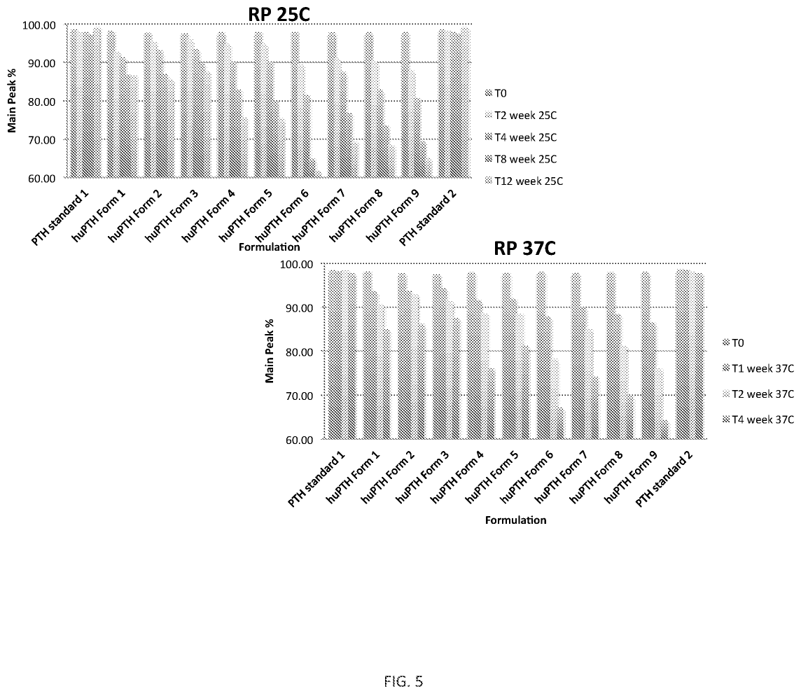

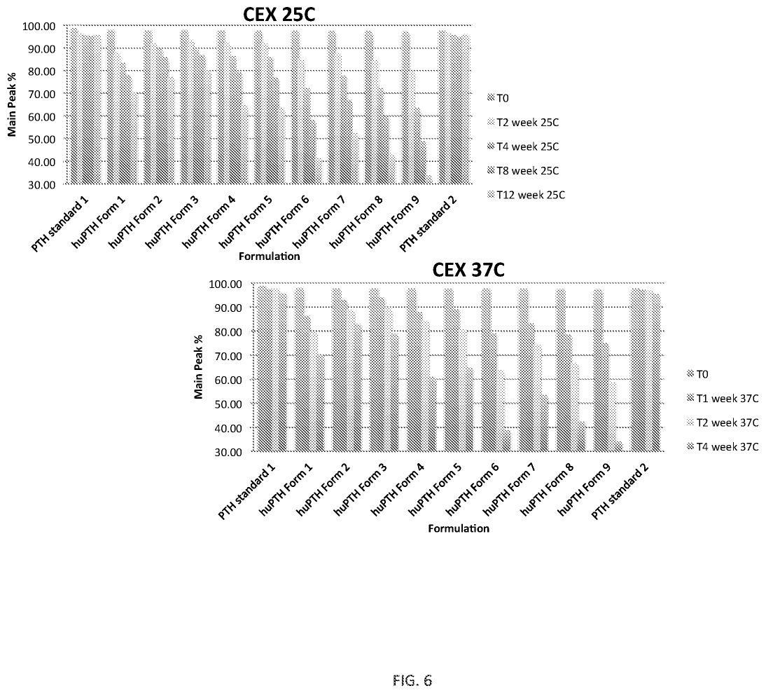

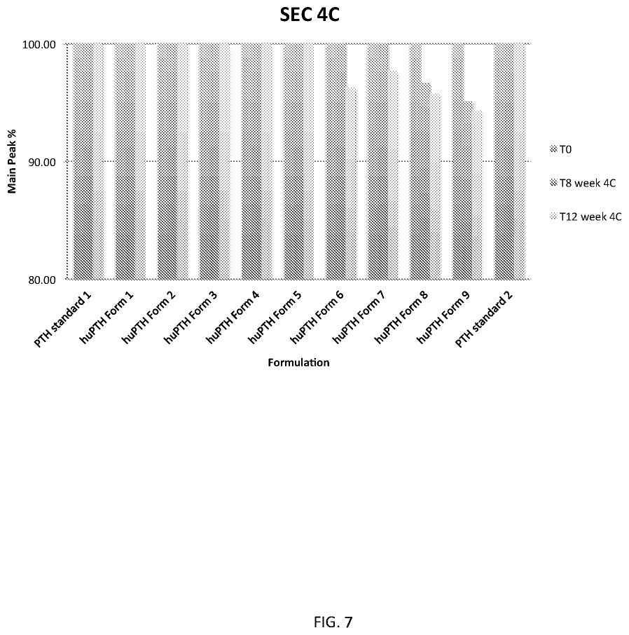

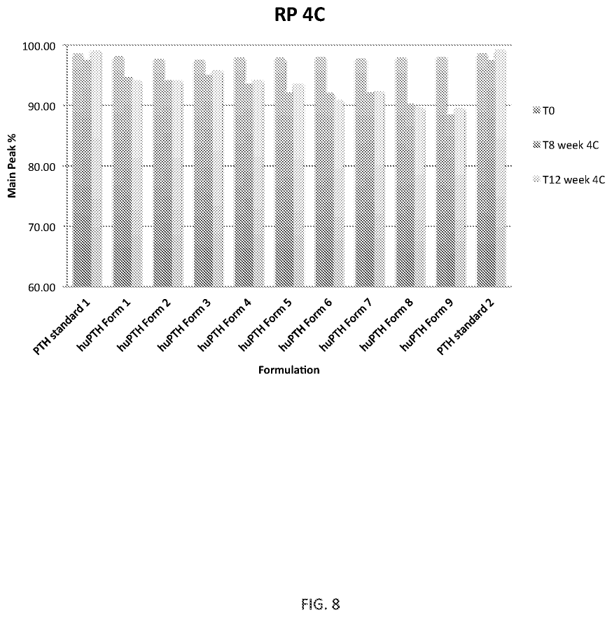

The results of SEC-HPLC, RP-HPLC and CEX-HPLC performed at 25° C. and 37° C. are depicted in FIGS. 4-6. The results of SEC-HPLC and RP-HPLC performed at 4° C. are depicted in FIGS. 7-8. These accelerated stability studies at 25° C. and 37° C. show that huPTH may have best stability profile at pH 5.

### EXAMPLE 3

### Screening Study for Excipients and Surfactants at Optimized pH Range

This example demonstrates the effects of various excipients and surfactants on the stability of liquid huPTH formulations at pH 5.0. Recombinant huPTH (1 mg/mL) having the amino acid sequence set forth in SEQ ID NO: 2 was used as an active ingredient in the formulations. The fixed formulation parameters were pH 5.0 and 1.0 mg/mL huPTH.

To screen the effects of various excipients, ten different formulations were prepared and stored in type 1 glass vials with TFE stoppers. Stability analysis (RP-HPLC, CEX-HPLC and SEC-HPLC, pH, Osmolarity, UV-VIS) was performed at 4° C., 25° C., 37° C. at 0, 1, 2, 4, 8, 12 weeks. Table 2 provides a summary of the formulations that were tested in this example.

TABLE 2
10 mM sodium citrate, 50 mM ZnCl₂, pH 5.0
10 mM sodium citrate, 10 mM L-Met, 50 mM ZnCl₂, pH 5.0
10 mM sodium citrate, 10 mM L-Met, 5% (w/v) Manitol, pH 5.0
10 mM sodium citrate, 10 mM L-Met, 5% (w/v) Sorbitol, pH 5.0
10 mM sodium citrate, 10 mM L-Met, 9% (w/v) Sucrose, pH 5.0
10 mM sodium citrate, 10 mM L-Met, 5% (w/v) Trehalose, pH 5.0
10 mM sodium citrate, 10 mM L-Met, 10% (v/v) Propylene Glycol,
pH 5.0
10 mM sodium citrate, 10 mM L-Met, 10% (v/v) Glycerol, pH 5.0
10 mM sodium citrate, 10 mM L-Met, 10% (w/v) Kolliphor P188, pH 5.0
10 mM sodium citrate, 10 mM L-Met, 100 mM NaCl, pH 5.0

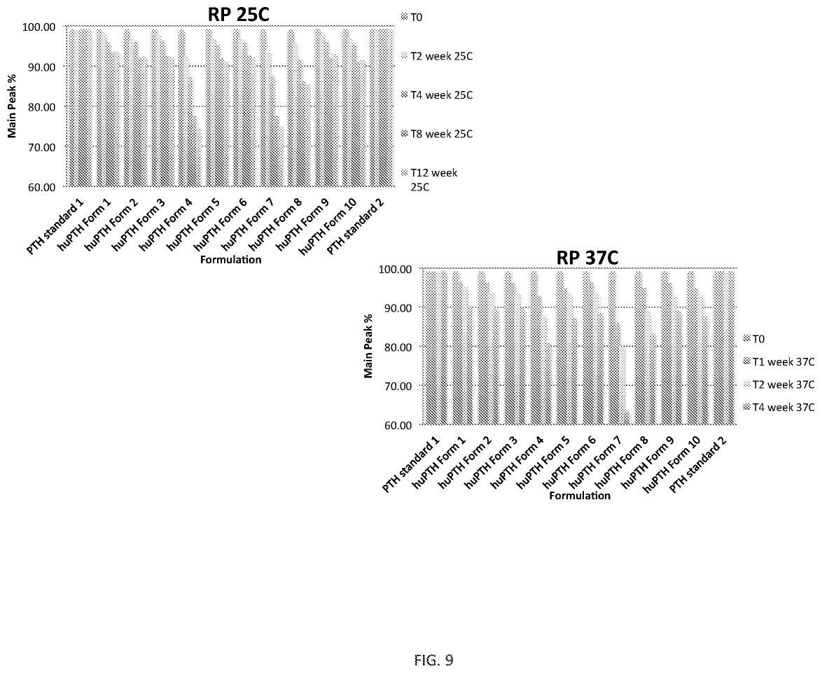

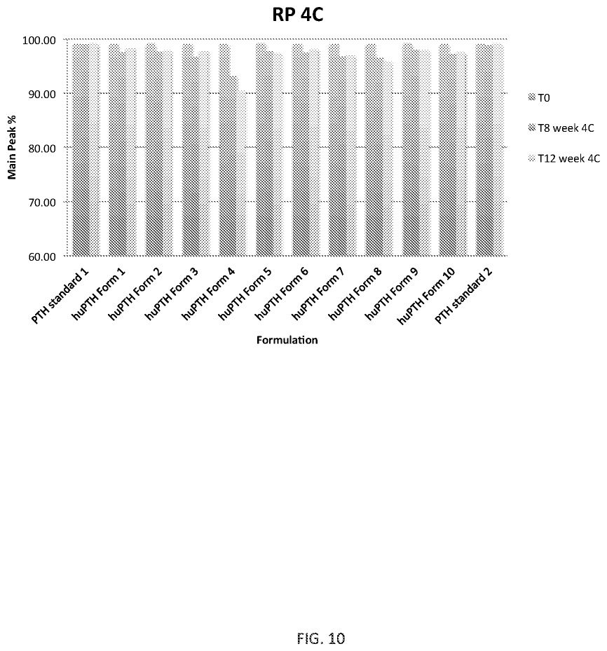

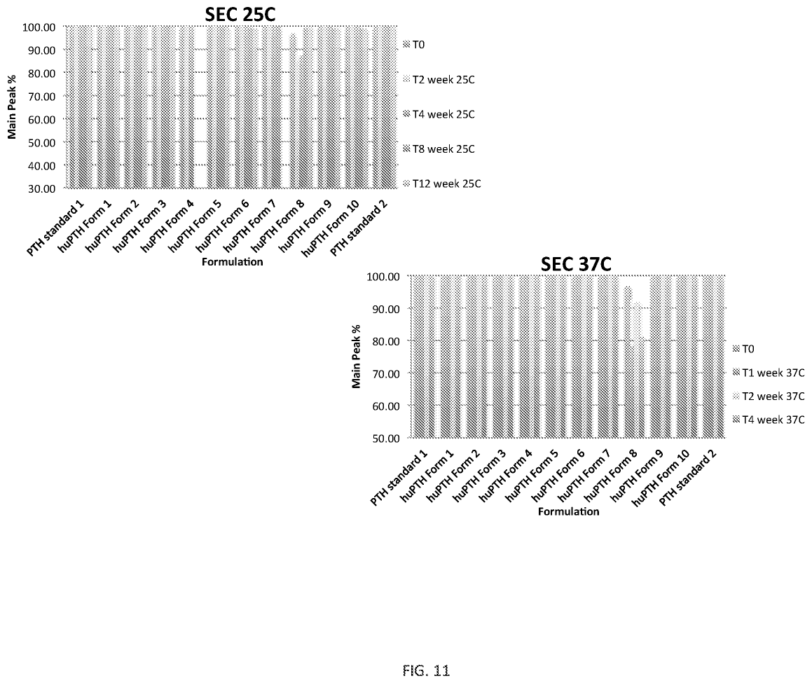

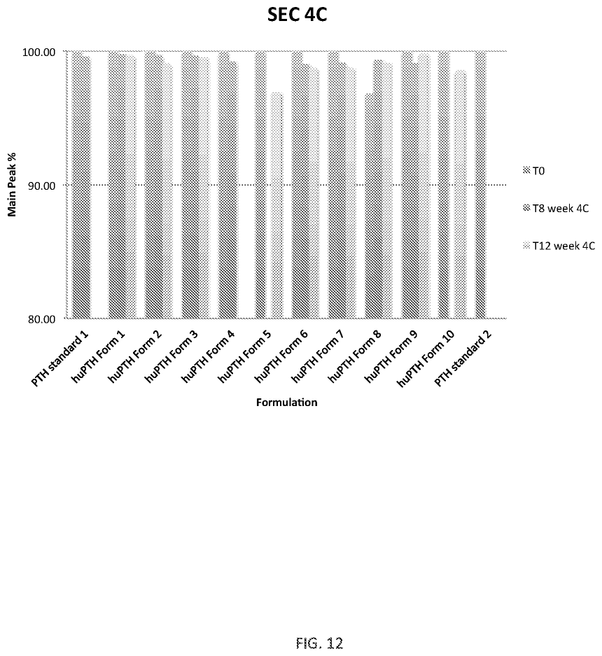

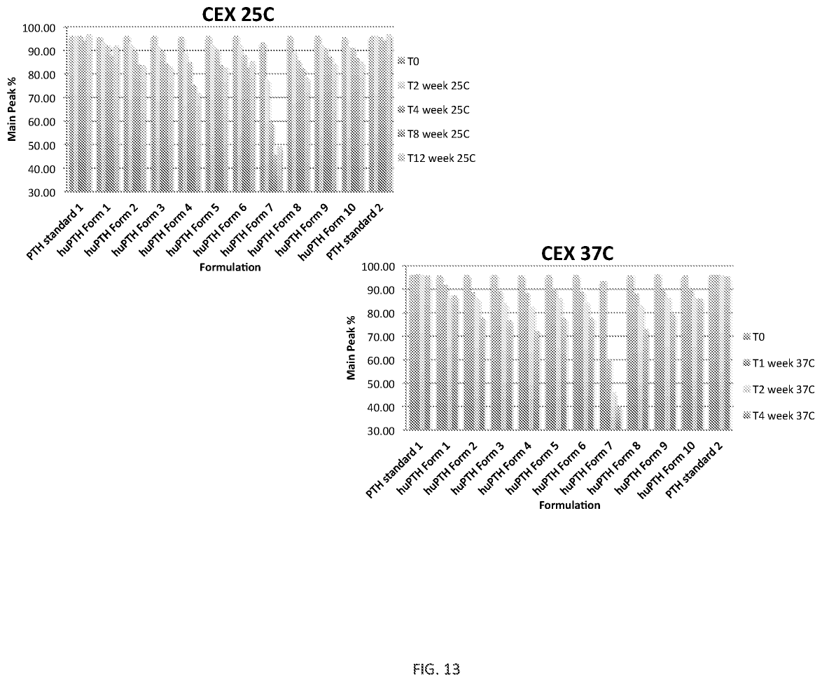

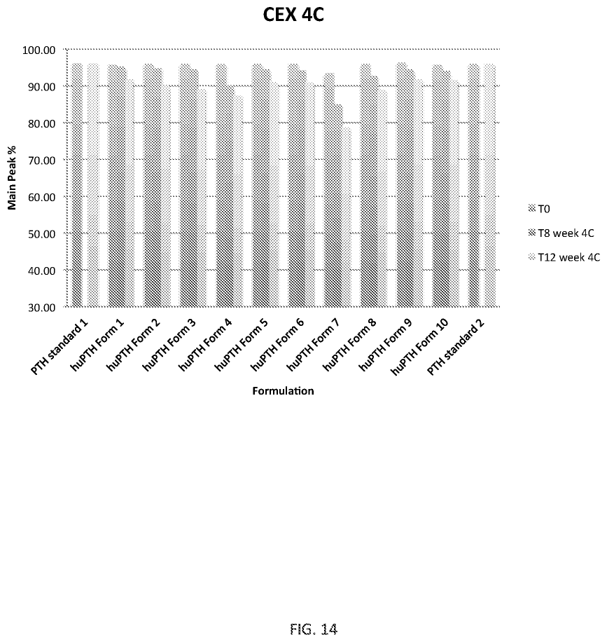

The results of SEC-HPLC, RP-HPLC and CEX-HPLC performed at 4° C., 25° C. and 37° C. are depicted in FIGS. 9-14. Initial accelerated stability studies at 25° C. and 37° C. show that stability of huPTH at pH 5 can be further increased in the presence of certain formulation excipients. Using ZnCl₂ as a bench mark, three formulation excipients provide better stability profile for huPTH in aqueous solutions and SEC-HPLC, RP-HPLC and CEX-HPLC data show that formulations #1, #2, #3, #5, #6, #9 and #10 are most stable under stressed accelerated conditions.

To screen the effects of various surfactants, twelve different formulations were prepared and stored in type 1 glass vials with TFE stoppers. Stability analysis (RP-HPLC, CEX-HPLC and SEC-HPLC, pH, Osmolarity, UV-VIS) was performed at 4° C., 25° C., 37° C. at 0, 1, 2, 4, 8, 12 weeks. Table 3 provides a summary of the formulations that were tested in this example.

TABLE 3
10 mM sodium citrate, 10 mM L-Met, 50 mM ZnCl₂, pH 5.0,
0.01% (v/v) polysorbate 20
10 mM sodium citrate, 10 mM L-Met, 5% (w/v) Sorbitol, pH 5.0,
0.01% (v/v) polysorbate 20
10 mM sodium citrate, 10 mM L-Met, 10% (v/v) Propylene Glycol,
pH 5.0, 0.01% (v/v) polysorbate 20
10 mM sodium citrate, 10 mM L-Met, 100 mM NaCl, pH 5.0, 0.01% (v/v)
polysorbate 20
10 mM sodium citrate, 50 mM ZnCl₂, pH 5.0, 0.01% (v/v)
polysorbate 20
10 mM sodium citrate, 10 mM L-Met, 50 mM ZnCl₂, pH 5.0,
0.1% (v/v) polysorbate 20
10 mM sodium citrate, 10 mM L-Met, 5% (w/v) Sorbitol, pH 5.0,
0.1% (v/v) polysorbate 20
10 mM sodium citrate, 10 mM L-Met, 5% (w/v) Sorbitol, pH 5.0,
0.5% (v/v) polysorbate 20
10 mM sodium citrate, 10 mM L-Met, 10% (v/v) Propylene Glycol,
pH 5.0, 0.1% (v/v) polysorbate 20
10 mM sodium citrate, 10 mM L-Met, 100 mM NaCl, pH 5.0, 0.1% (v/v)
polysorbate 20
10 mM sodium citrate, 50 mM ZnCl₂, pH 5.0, 0.1% (v/v)
polysorbate 20
10 mM sodium citrate, 10 mM L-Met, 100 mM NaCl, pH 5.0, 0.1% (v/v)
Kolliphor P188

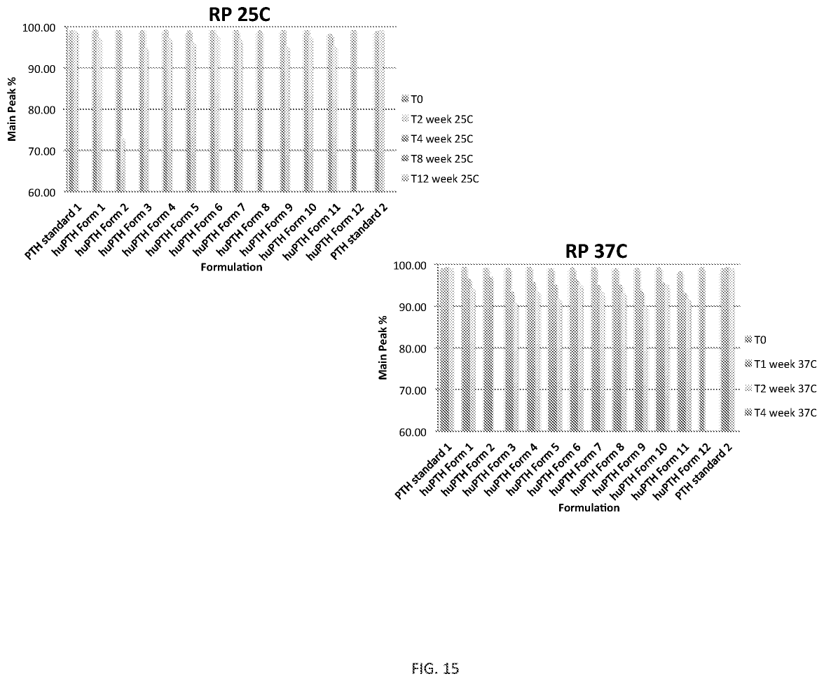

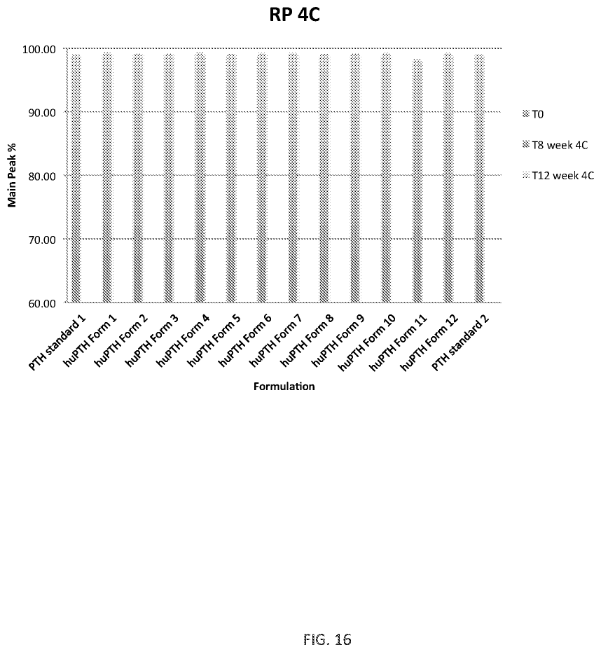

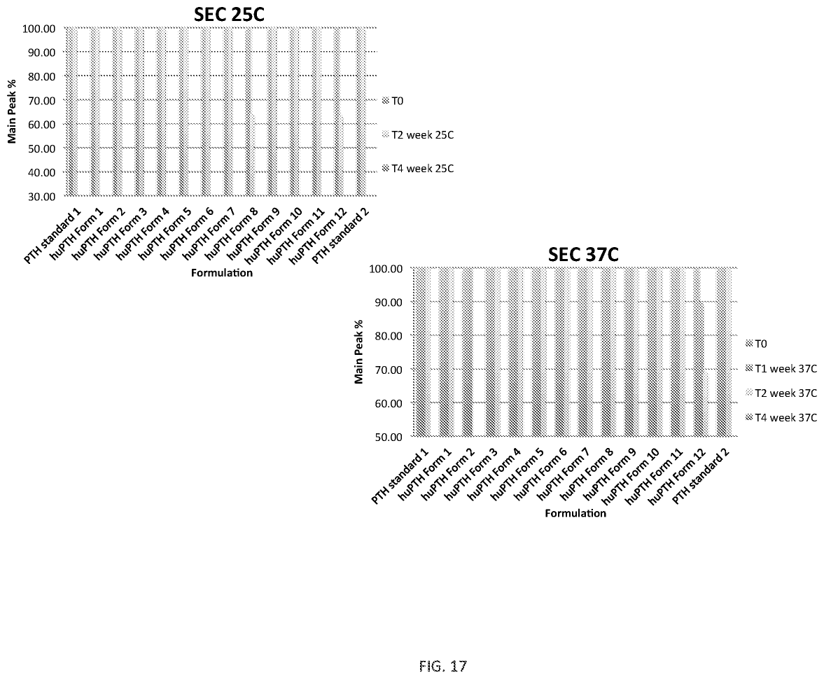

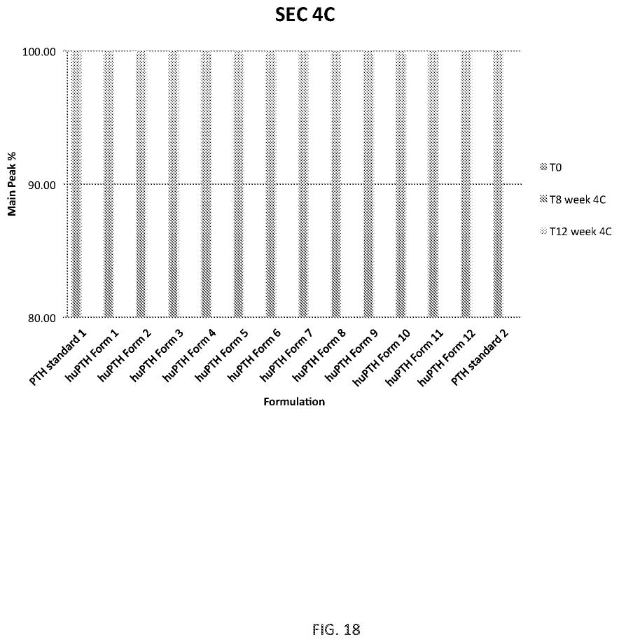

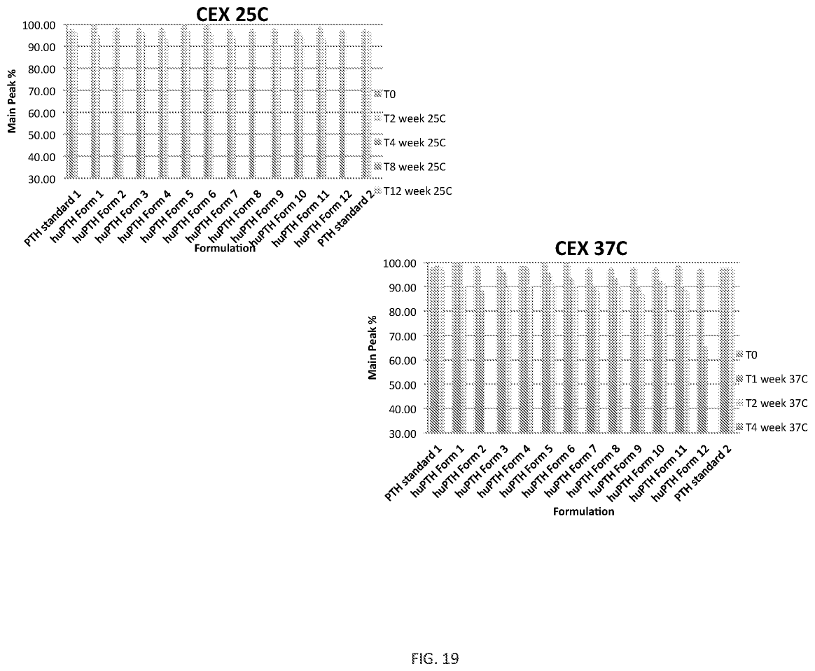

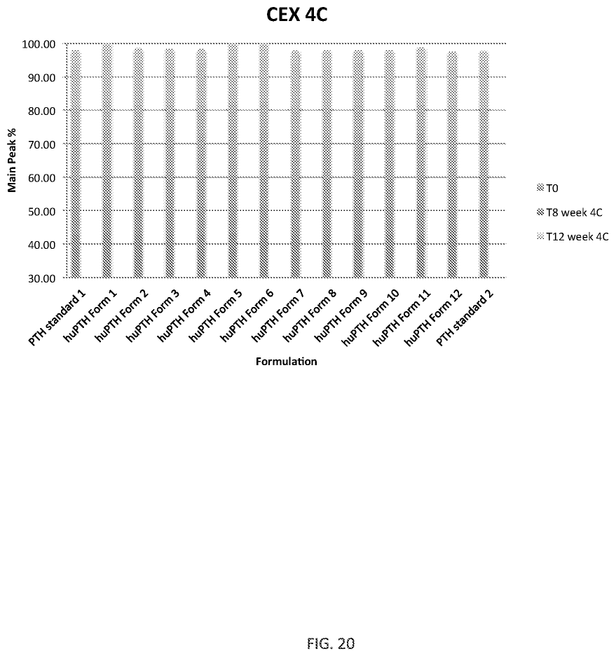

The results of SEC-HPLC, RP-HPLC and CEX-HPLC performed at 4° C., 25° C. and 37° C. are depicted in FIGS. 15-20. RP-HPLC data (25° C.) show that formulations #1, #3, #4, #5, #6, #7, #9, #10, and #11 are best under stressed accelerated conditions whereas formulations #2, #8 and #12 are not stable. SEC-HPLC data (25° C.) show that all formulations except #8 and #12 are stable. CEX-HPLC data show that all formulations except #2, #8 and #12 are stable.

It was thus demonstrated that combination of L-Met, propylene glycol, and polysorbate-80 can provide good stability for huPTH at pH 5.0. And with the exception of formulations #2, #8 and #12, all formulations are stable as of two weeks. This L-Met effect on huPTH stability is a novel finding.

### EXAMPLE 4

### Effect of Tween 20 and Tween 80 on the stability of huPTH

This example evaluated the effects of Tween 20 (T20) or Tween 80 (T80) on the stability of various formulations tested in Examples 1-3. Table 4 provides a summary of the formulations that were initially tested in this example.

TABLE 4

0.01% T20, 150 mM NaCl, 10 mM NaOAC, pH 5.0
0.1% T20, 150 mM NaCl, 10 mM NaOAC, pH 5.0
0.01% T80, 150 mM NaCl, 10 mM NaOAC, pH 5.0
0.1% T80, 150 mM NaCl, 10 mM NaOAC, pH 5.0

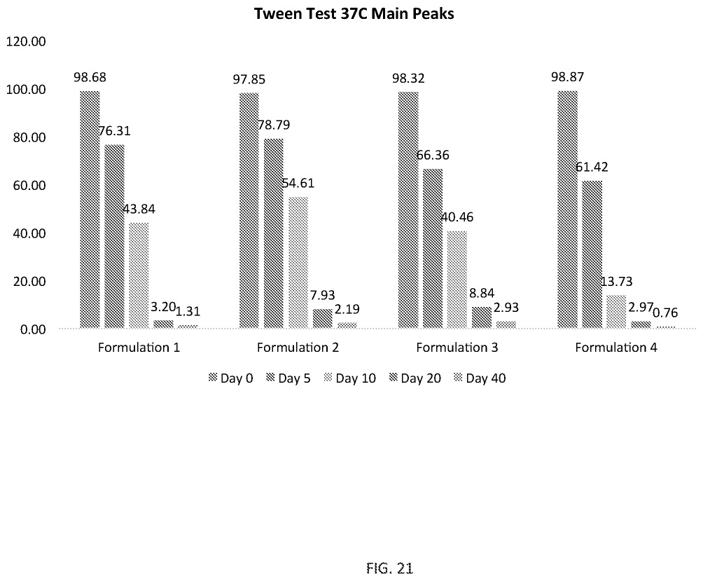

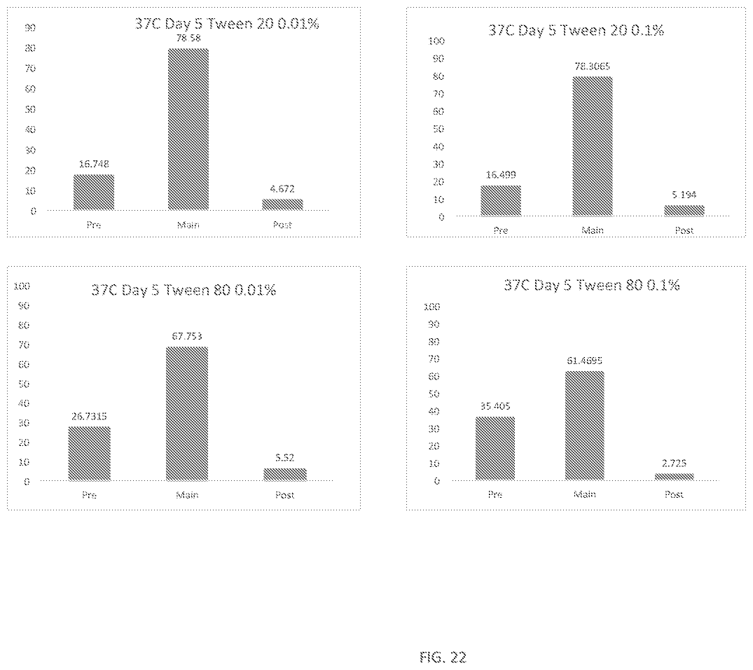

The results of RP-HPLC performed at 37° C. is depicted in FIG. 21. The stability of the formulations at 37° C. after 5 days is depicted in FIG. 22. The results demonstrated that Tween 80 caused more degradation in PTH than Tween 20. In fact, 0.01% (v/v) Tween 80 caused more degradation than 0.1% (v/v) Tween 20. The observed degradations are primarily oxidations.

In a second study, the formulations listed in Table 5 were evaluated.

TABLE 5
150 mM NaCl, 10 mM methionine, 10 mM NaOAc, 0.01% Tween 20
150 mM NaCl, 10 mM methionine, 10 mM NaOAc, 0.01% Tween 80
100 mM NaCl, 50 mM methionine, 10 mM NaOAc, 0.01% Tween 20
100 mM NaCl, 50 mM methionine, 10 mM NaOAc, 0.01% Tween 80
50 mM NaCl, 100 mM methionine, 10 mM NaOAc, 0.01% Tween 20
50 mM NaCl, 100 mM methionine, 10 mM NaOAc, 0.01% Tween 80

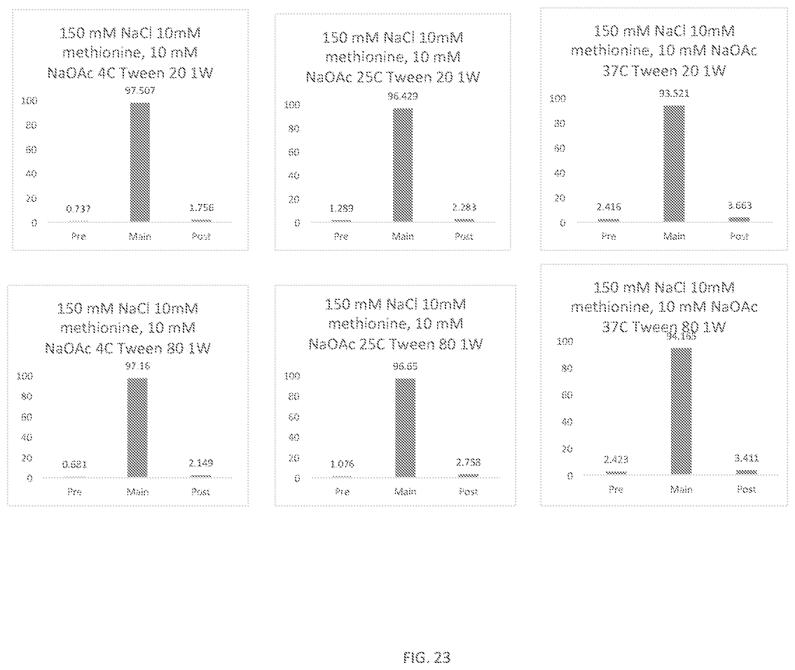

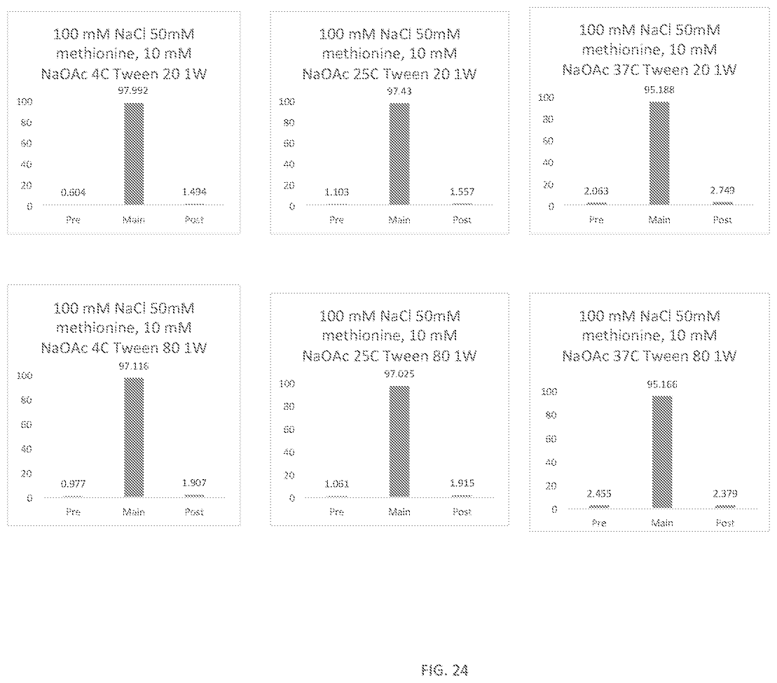

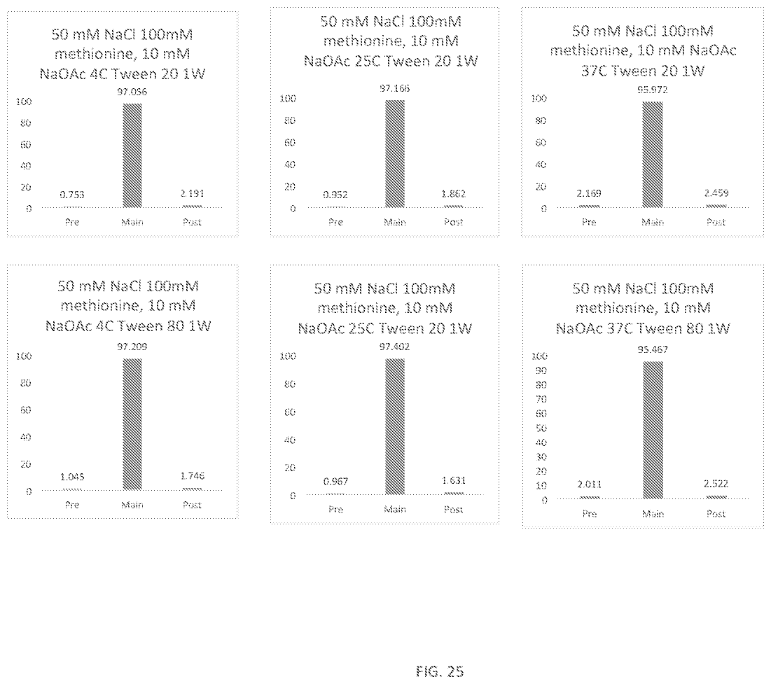

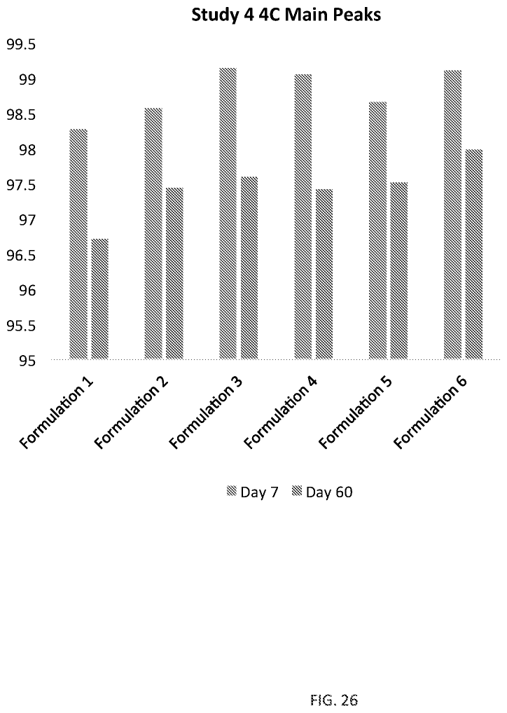

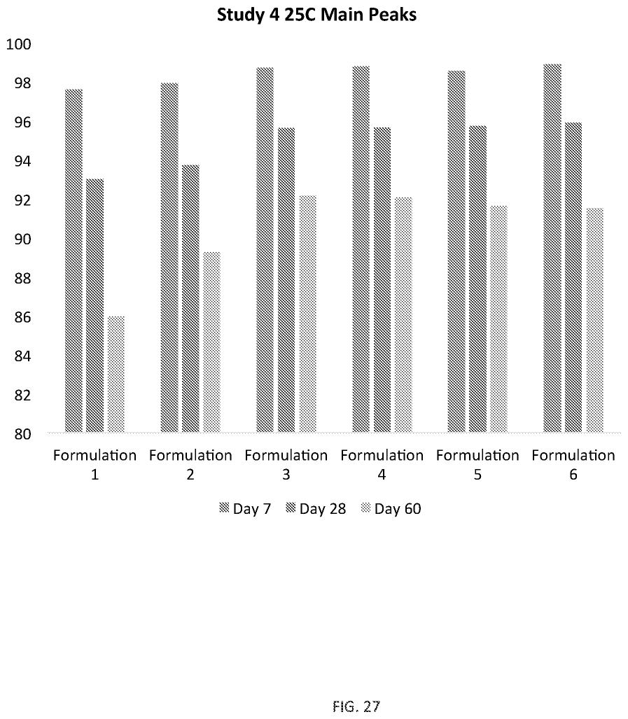

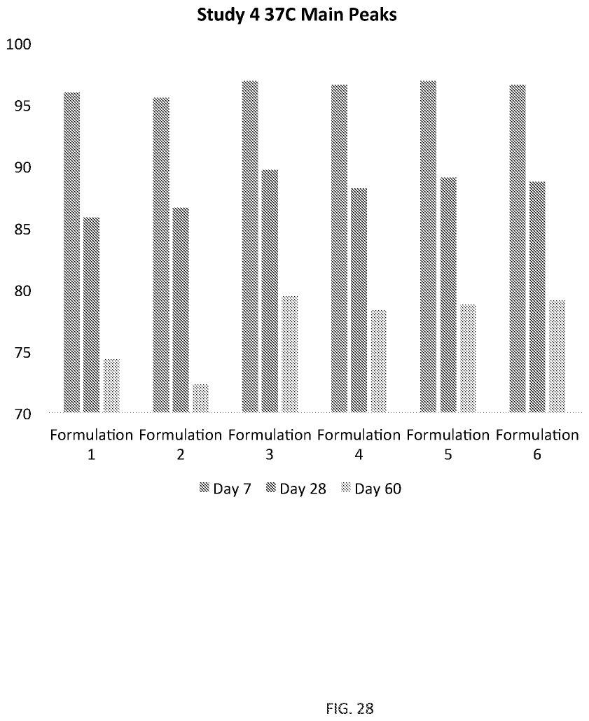

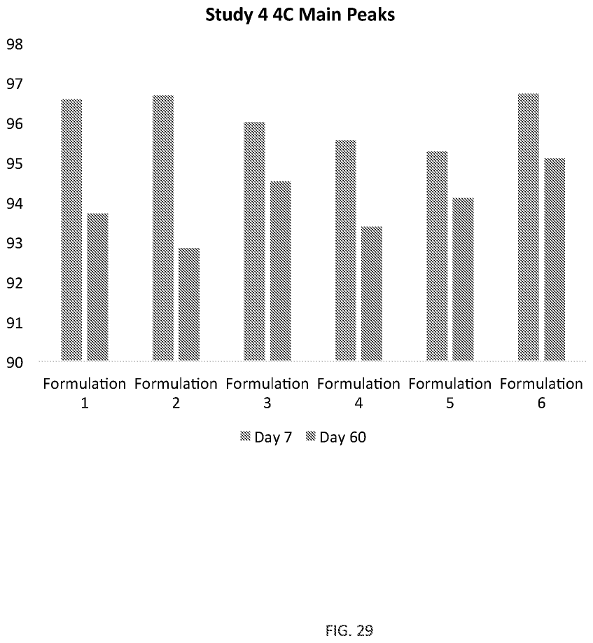

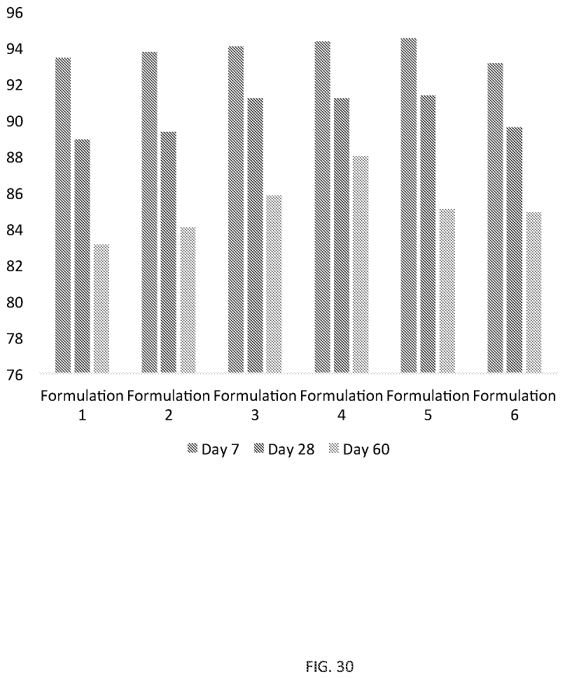

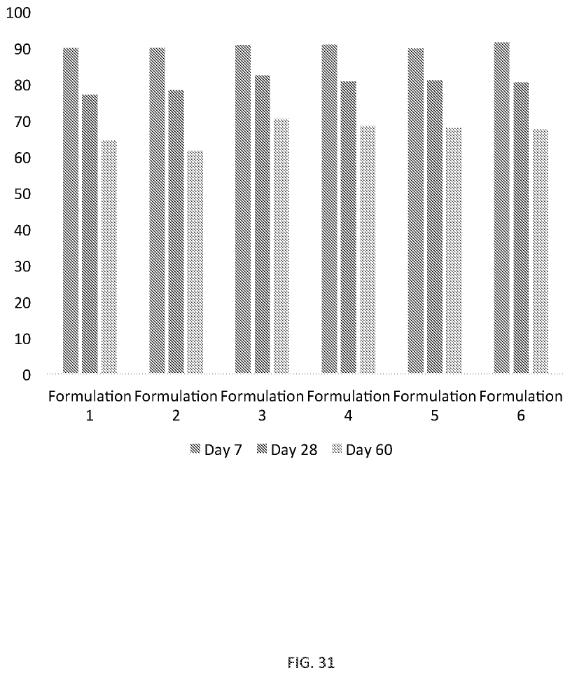

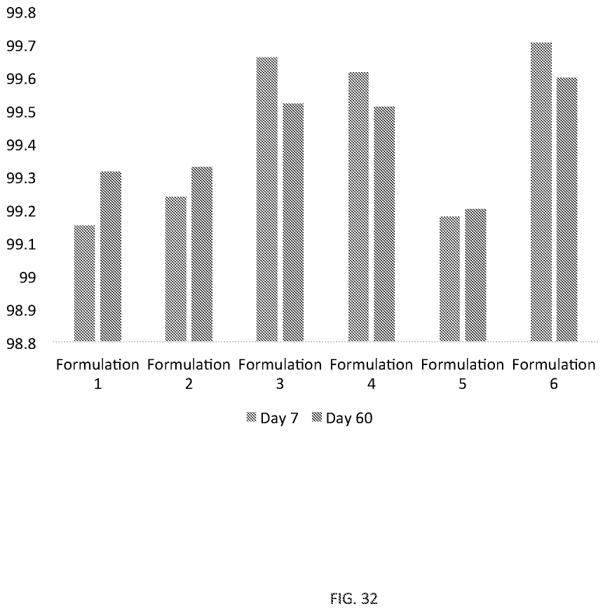

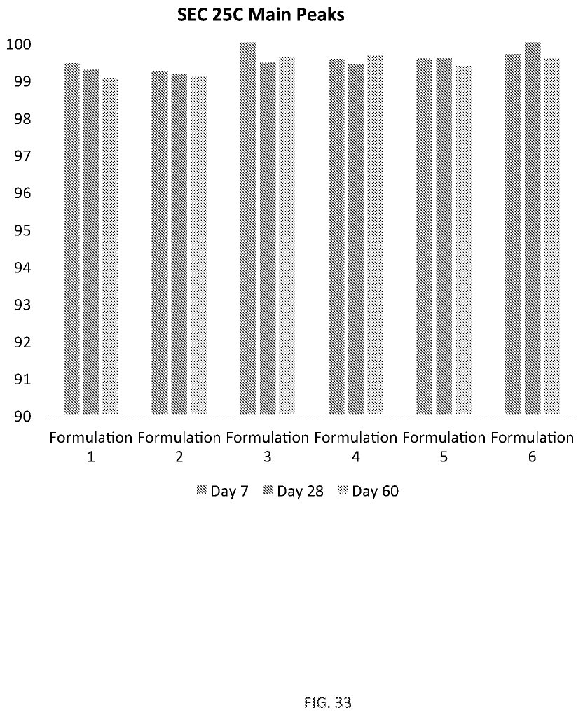

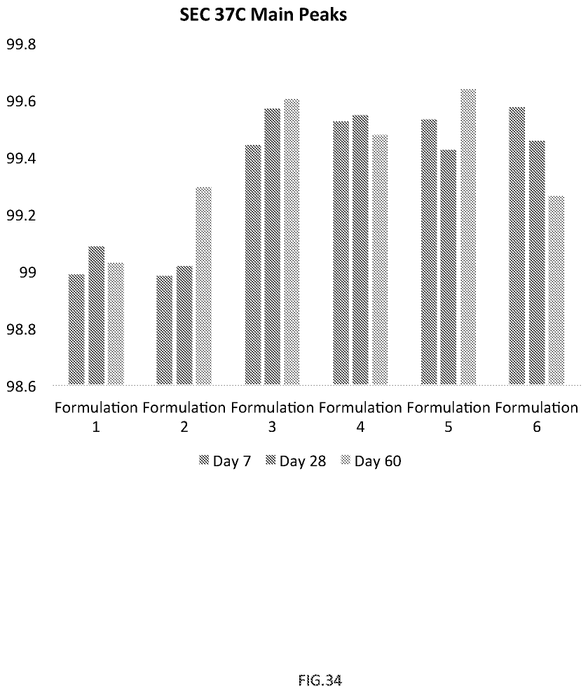

The stability of the formulations at 4° C., 25° C. and 37° C. at 1 week is depicted in FIGS. 23-25. The results of RP-HPLC, CEX-HPLC and SEC-HPLC performed at 4° C., 25° C. and 37° C. are depicted in FIGS. 26-34. These results demonstrate that L-Met concentration needs to be increased from 10 mM to 50-100 mM in order to prevent Tween 80 induced oxidation and the increase in the concentration of L-Met leads to protective effect of huPTH stability and at above 50 mM, the protective effect of L-Met appears to be maximized.

### EXAMPLE 5

### Evaluation of L-Met and EDTA Combinations on the Stability of huPTH

This example evaluated the effects of various combinations of L-Met and EDTA on the stability of huPTH. Table 6 provides a summary of the formulations that were initially tested in this example.

TABLE 6

10 mM sodium citrate, 50 mM L-Met, 5 mM EDTA, pH 5.0,
4% Sorbitol 0.01% polysorbate 20
10 mM sodium citrate, 50 mM L-Met, 5 mM EDTA, pH 5.0,
100 mM NaCl, 0.01% polysorbate 20
10 mM sodium citrate, 50 mM L-Met, 10 mM EDTA, pH 5.0,
4% Sorbitol 0.01% polysorbate 20
10 mM sodium citrate, 50 mM L-Met, 10 mM EDTA, pH 5.0,
100 mM NaCl, 0.01% polysorbate 20
10 mM sodium citrate, 50 mM L-Met, 10 mM EDTA, pH 5.0,
100 mM NaCl, 0.01% polysorbate 80
10 mM sodium citrate, 5 mM EDTA, pH 5.0, 4% Sorbitol,
0.01% polysorbate 20
10 mM sodium citrate, 5 mM EDTA, pH 5.0, 100 mM NaCl,
0.01% polysorbate 20
10 mM sodium citrate, 10 mM EDTA, pH 5.0, 4% Sorbitol,
0.01% polysorbate 20
10 mM sodium citrate, 10 mM EDTA, pH 5.0, 100 mM NaCl,
0.01% polysorbate 20
10 mM sodium citrate, 150 mM L-Met, pH 5.0,
0.01% polysorbate 20
10 mM sodium citrate, 150 mM L-Met, 5 mM EDTA, pH 5.0,
0.01% polysorbate 20

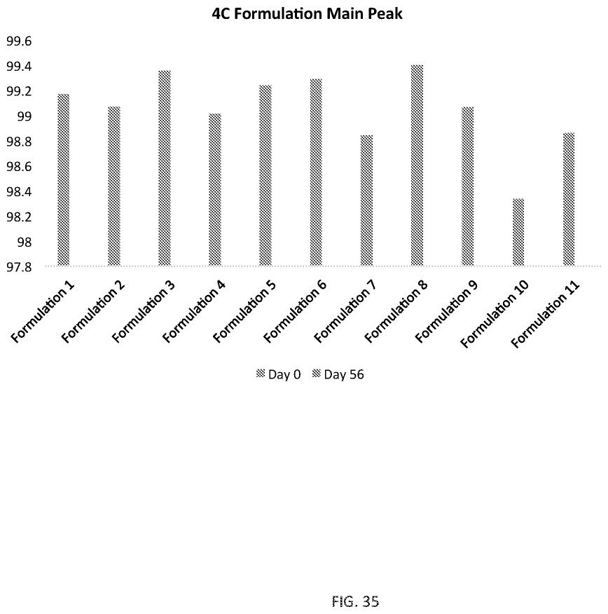

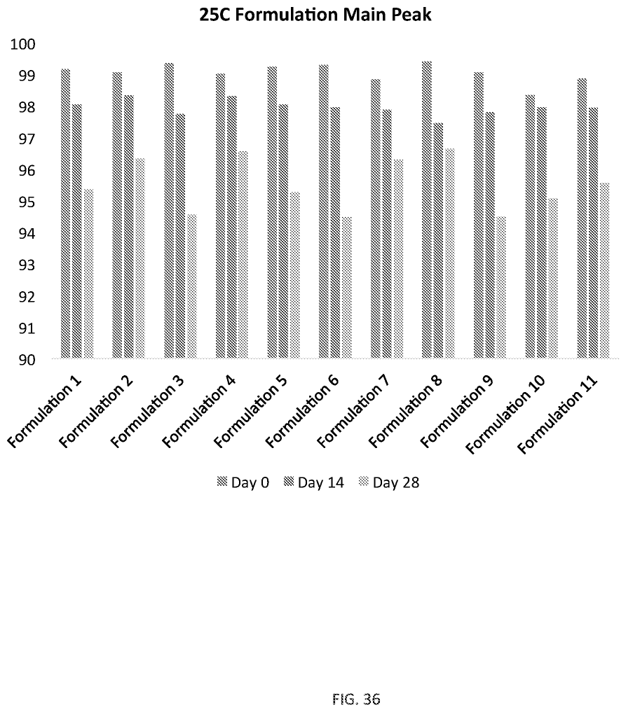

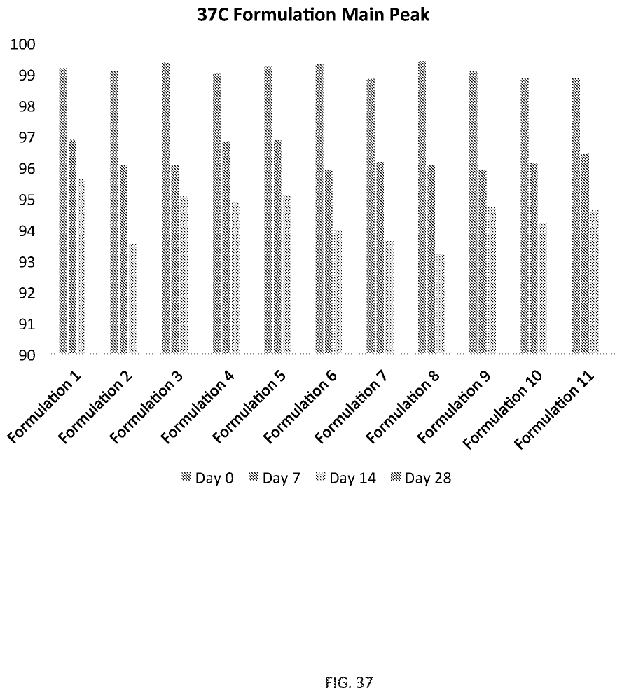

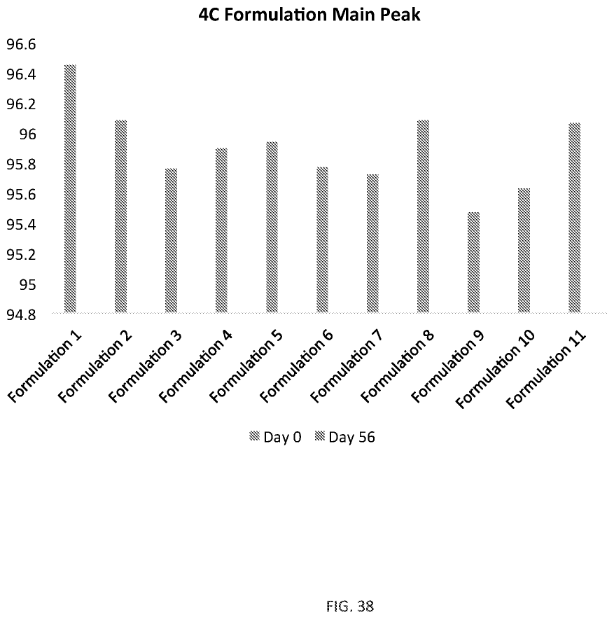

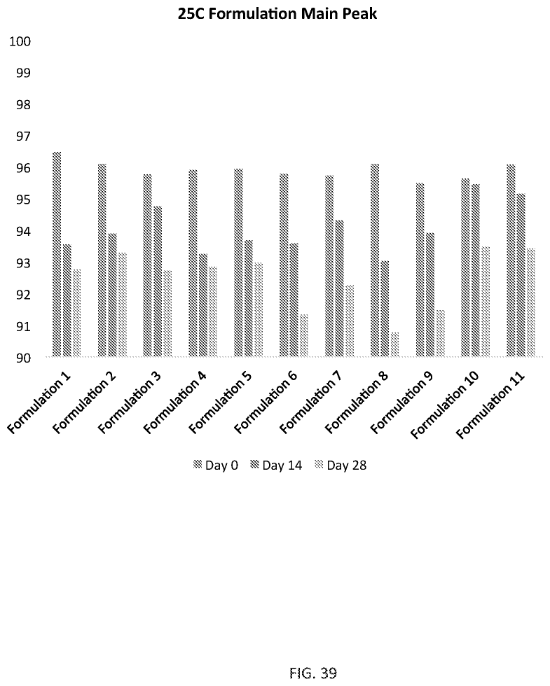

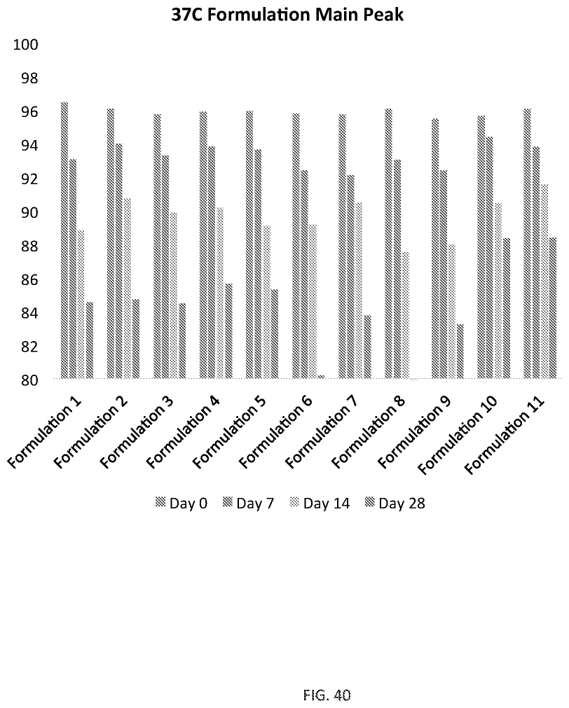

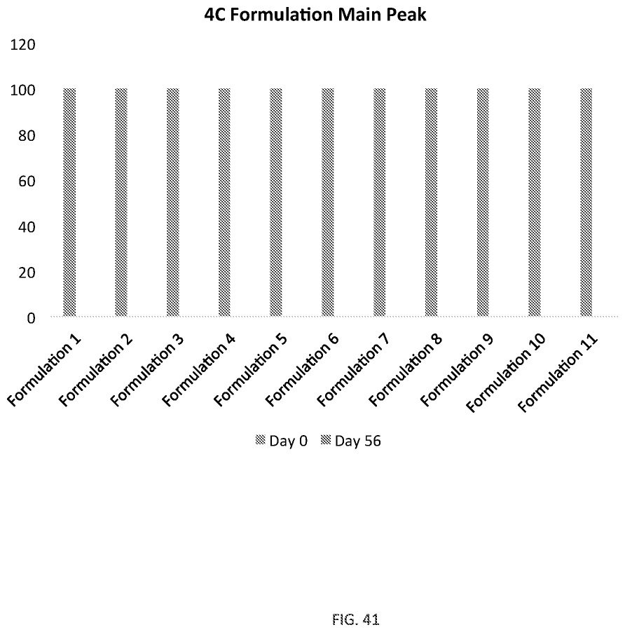

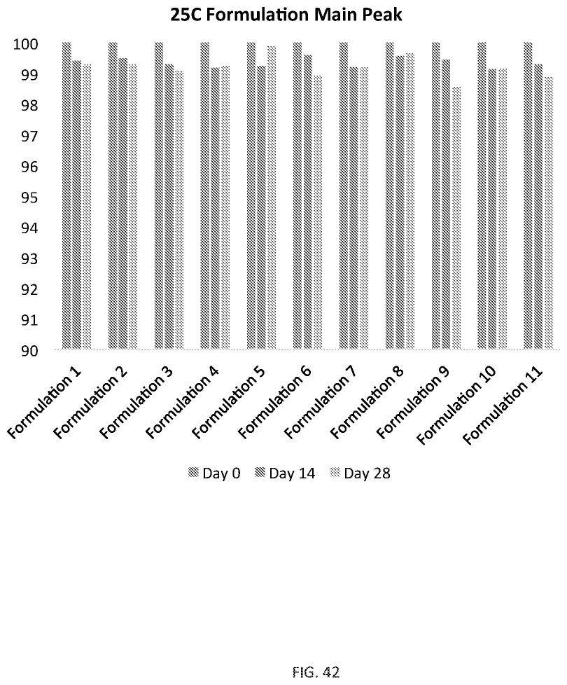

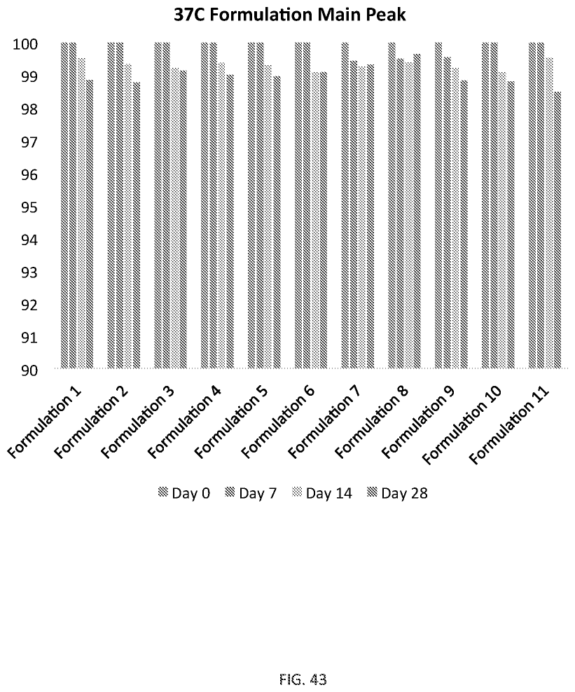

The results of RP-HPLC, CEX-HPLC and SEC-HPLC performed at 4° C., 25° C. and 37° C. are depicted in FIGS. 35-43. These results demonstrate that the optimal formulation comprises 10 mM sodium citrate, 50 mM L-Met, 10 mM EDTA, pH 5.0, 100 mM NaCl, 0.01% polysorbate 20. And, surprisingly, it appears that combination of sorbitol and EDTA can stabilize huPTH at 25° C. Based on the findings described herein, potential process parameters can be as following: dilute poloysorbate 20 into formulation buffer to make 10% (v/v) concentration; dialyze huPTH against formulation buffer to make drug substance (DS); spike 10% (v/v) of polysorbate 20 into drug substance (DS) to make drug product (DP).

### Additional Methods

### SEC-HPLC

SEC Column: Phenomenex Yarra 3 μm SECd200 LC Column, 300×4.6 mm, s/no H15d152245
Mobile phase: 0.3M NaCl, 50 mM NaH₂PO₄ pH 7.0
Flow rate: 0.3 ml/min
Detector: 220 nm
Col. Temp: 25±3° C.
Auto Sampler: 5±2° C.
Injection: 20 μl of 1mg/ml protein

### RP-HPLC

RP Column: Phenomenex Jupiter 5p C18 300A, Column 250×4.6 mm, s/no 172611
Mobile phase A: 0.1% TFA, 99.9% ddH₂O
Mobile phase B: 0.1% TFA, 99.9% ACN
Flow rate: 0.5 ml/min
Detector: 220 nm
Col. Temp: 25±3° C.
Auto-Sampler: 5±2° C.
Injection: 20 μl of 1 mg/ml protein

### CEX-HPLC

CEX Column: TOSOH Bioscience, LLC TSKgel SP-NPR Column, 4.6 ID×3.5cm, 2.5 μm, s/no
K0054-81C
Mobile phase A: 38.75 mM NaAc, 22.5% ACN pH 5.5
Mobile phase B: 46.25 mM NaAc, 277.5 mM NaCl, 7.5% ACN pH 5.1
Flow rate: 0.3 ml/min
Detector: 220 nm
Col. Temp: 25±3° C.
Auto-Sampler: 5±2° C.
Injection: 20 μl mg/ml protein

What we claim is:

1. An aqueous pharmaceutical formulation comprising: (a) a therapeutically effective amount of a human parathyroid hormone (PTH); (b) a buffering agent to maintain the pH range of the solution from 3 to 6; (c) an effective amount of one or more stabilizing agents; (d) a tonicity modifier; and (e) the balance being water, wherein said solution is sterile and ready for parenteral administration to a human patient.

2. The formulation of claim 1, wherein the one or more stabilizing agents is selected from the group consisting of sugars, salts, surfactants, proteins, chaotropic agents, lipids, and amino acids.

3. The formulation of claim 1, wherein the one or more stabilizing agents is a polyol selected from the group consisting of mannitol, glycerol, sorbitol, inositol and a polyhydric alcohols, and propylene glycol or mixtures thereof.

4. The formulation of claim 2, wherein the one or more stabilizing agents is a non-ionic surfactant selected from the group consisting of polysorbate 20, polysorbate 40, polysorbate 60 and polysorbate 80.

5. The formulation of claim 2, wherein the one or more stabilizing agents is an amino acid selected from the group consisting glycine, alanine, serine, aspartic acid, glutamic acid, threonine, tryptophan, lysine, hydroxy lysine, histidine, arginine, cystine, cysteine, methionine, phenylalanine, leucine, isoleucine amino acids and their derivatives.

6. The formulation of claim 5, wherein the amino acid is L-methionine.

7. The formulation of claim 1, wherein the one or more stabilizing agents is a chelator selected from the group of EDTA, DTPA and EGTA.

8. The formulation of claim 1, wherein the tonicity modifier is selected from the group consisting of sodium chloride, potassium chloride, mannitol, sucrose, dextrose, sorbitol, and glycerin.

9. A formulation according to claim 1, wherein the human PTH comprises the amino acid sequence set forth in SEQ ID NO: 1.

10. A formulation according to claim 1, wherein the human PTH comprises the amino acid sequence set forth in SEQ ID NO: 2.

11. A formulation according to claim 1, wherein the formulation has a pH of about 4.5 to 5.5.

12. The formulation according to claim 1, wherein the human PTH is present in the formulation at a concentration of 0.1 mg/mL to 10 mg/mL.

13. The formulation according to claim 1, which further comprises a parenterally acceptable preservative.

14. A stable aqueous formulation comprising 1 mg/mL human PTH, 10 mM sodium citrate, 50 mM L-Met, 10 mM EDTA, 100 mM NaCl, 0.01% polysorbate 20, wherein the formulation has a pH of about 5.0.
